(19) United States
(12) Patent Application Publication
(10) Pub. No.: US 2022/0395289 A1
(43) Pub. Date: Dec. 15, 2022

(54) OPTICALLY GUIDED SUPRAPUBIC CYSTOSTOMY

(71) Applicant: Swan Valley Medical Incorporated, Bigfork, MT (US)

(72) Inventors: Ronald E. ZOOK, Bigfork, MT (US); Timothy E. BRAUN, Kalispell, MT (US); Paul BUREK, Livermore, CO (US)

(21) Appl. No.: 17/804,040

(22) Filed: May 25, 2022

Related U.S. Application Data
(60) Provisional application No. 63/268,781, filed on Mar. 2, 2022.
(60) Provisional application No. 63/192,936, filed on May 25, 2021.

(51) Int. Cl. A61B 17/32

(52) U.S. Cl. CPC A61B 17/320016; A61B 2218/002; A61B 2017/00274

(57) ABSTRACT

A medical instrument and associated functionality are described for performing a cystostomy with optical guidance. A cystostomy device (10) includes a sound (12) including a distal portion (18) and a proximal portion (20), a front handle (14) and a rear handle (16). A physician or other user can grip the handles (14 and 16) to guide the distal portion (18) of the sound (12) through the urethra and into a bladder of the patient. The device (10) includes an optical unit (32) mounted at the tip (22) of the sound (12). The optical unit (32) generally includes a distal sound tip cap (34) with a lens opening and an optical lens (36) mounted on the opening. The optical unit (32) may further include one or more illumination sources, such as LEDs, for illuminating a volume forward of the tip (22) of the sound (12).

### CROSS-REFERENCE TO RELATED APPLICATION

This application claims benefit of provisional U.S. Patent Application No. 63/192,936 entitled “OPTICALLY GUIDED TRANSURETHRAL SUPRAPUBIC CYSTOSTOMY” filed on May 25, 2021. This application also claims priority to provisional U.S. Patent Application No. 63/268,781, entitled, “OPTICALLY GUIDED TROCAR INSERTION FOR SUPRAPUBIC CYSTOSTOMY”, filed on Mar. 2, 2022. The contents of the above-noted applications (collectively, the “parent applications”) are incorporated by reference herein as if set forth in full and priority to these applications are claimed to the full extent allowable under U.S. law and regulations.

### FIELD OF THE INVENTION

The present invention relates generally to bladder aspiration procedures such as percutaneous or transurethral suprapubic cystostomy and, in particular, to providing illuminated optical guidance and physiological examination for such procedures. Such procedures include opening a pathway to a patient's bladder (e.g., to insert a catheter) via both inside-out approaches (“Transurethral Suprapubic-endo Cystostomy”), where the pathway is formed from inside the bladder to outside the patient's abdominal wall, and outside-in approaches, e.g., via a percutaneous trocar punch (“Suprapubic Percutaneous Cystostomy”) or via a percutaneous incision utilizing a cannula/obturator with cutting blade. Optically guided outside-in procedures and optically guided inside-out procedures are described in this application.

### BACKGROUND OF THE INVENTION

Transurethral procedures are commonly used to diagnose and/or treat a number of medical conditions. One such procedure is placing a suprapubic catheter for treatment of bladder dysfunction due to urinary retention, incontinence and for fluid management of the hospitalized patient. To place the suprapubic catheter, a surgical pathway is first opened between the patient's bladder and the abdominal wall of the patient. This pathway is generally opened by forming a narrow incision, widening the incision pathway, and inserting the catheter via the widened pathway. The initial percutaneous access may take the form of an incision with a cutting blade or may be formed from the outside-in via a percutaneous trocar (hollow needle) punch. However, there is a significant risk of complications, e.g., associated with perforating the patient's bowels, in connection with an outside-to-inside blind percutaneous access through either a cutting blade with cannula, needle or trocar punch method.

The Transurethral Suprapubic endo Cystostomy (T-SPeC®) procedure, developed by Swan Valley Medical Incorporated, which is the world's only inside-to-outside suprapubic placement device, has gained clinical acceptance for dramatically reducing risks, including injury and mortality, associated with suprapubic cystostomy performed with other outside-to-inside instruments, such as percutaneous trocar punch procedures. The T-SPeC® procedure is initiated transurethrally and performed with the aid of the design of the T-SPeC® device, providing for physical anatomical feedback allowing for proper positioning of the T-SPeC® sound within the bladder, at the dome of the bladder, adjacent to the pubic symphysis before deploying the cutting blade from inside-to-outside of the bladder through the abdomen. This is the only safe location for exiting the bladder and is currently located by anatomical tactile feedback from the T-SPeC® instrument. Once the deployment of the T-SPeC® cutting blade is extended from inside the instrument, located inside the bladder, to outside of the bladder and abdominal wall, the cutting blade is detached, and a urinary catheter is attached and withdrawn back into bladder. Though no serious complications have been reported, if the instrument is not passed through the urethral and placed exactly in the correct location, serious injury to the patient or even death is possible due to urethral perforation or bowel injury. While such complications generally have been avoided with the T-SPeC® instrument as discussed below, given the movement to involve less skilled practitioners in many procedures, there is a need for improved certainty of safe operation as well as ease of use.

The T-SPeC® device was designed to replace the existing percutaneous (outside-to-inside) suprapubic catheterization placement techniques, as the percutaneous trocar punch devices, the predominant current method used in the market, have high mortality and morbidity, (4.4% mortality, 45% complication rate), as well as open cystostomy (1.8% mortality and 30% complication rate). The T-SPeC® device has proven to be extremely safe in the hands of trained urologists performing the procedure. Since market introduction in 2013 in the U.S., E.U., Canada and Mexico, there have been no reportable adverse outcomes and no instrument failures with thousands of procedures performed.

Efforts are now focusing on the replacement of urethral catheterization with suprapubic catheterization in the hospitalized patient environment, with a primary initial focus on the critical care (CCU) patient population, which historically receives urethral catheterization for over 70% of the CCU patient population. Over 25% of the general admitted hospitalized inpatient population receive urethral catheters. Because of the proven clinical benefits of suprapubic catheterization, which include elimination of common serious complications due to urethral catheterization placement, including urethral perforation, prostate and bladder sphincter injuries, high infection rates etc., suprapubic catheterization is now available to be placed safely, due to the introduction of the T-SPeC® instrument, the only inside-to-outside procedure, to avoid these serious infections and complications associated with urethral catheterization. However, this device is contraindicated for pediatric patients and patients with an occluded urethra due to stricture disease (urethral scarring primarily due to prolonged use of indwelling urethral catheterization or benign prostatic hypertrophy (BPH).

The T-SPeC® procedure has become the enabling technology for replacing urethral catheterization with suprapubic catheterization, saving hospitals millions of dollars annually with this change in clinical practice, due to the avoidance of serious complications with urethral catheterization and consequential extended length of stay. The T-SPeC® procedure begins with the “blind” entry to the meatus, navigation through the urethra, past the prostate gland (male), through the bladder sphincter and into the bladder. Prior to initiating a cystostomy, an endoscope/cystoscope is used to inspect the health of the urethra passage, a procedure called a cystoscopy. Urologists are very familiar with these procedures. Also, urologists may perform a cystoscopy to examine the bladder for presence of bladder cancer, a contraindication for a cystostomy procedure.

Due to the acute shortage of urologists and because the associated practice is primarily a physician office practice, urologists cannot support the initiative to place suprapubic catheters in hospitalized patients. Over 70% of all critical care patients are transferred from the emergency department (E.D.), with the remainder, post-operative patients coming from Med/Surg treatment. In order to place suprapubic catheters in patients being transferred to the CCU, it is necessary to provide T-SPeC® procedure training to emergency physicians and mid-level clinicians such as PAs and registered nurse practitioners.

The T-SPeC® procedure is evolving from the typical urologist's practice and their typical urologic patient towards the hospitalized medical/surgical patients, emergency/trauma patients and critical care patients which requires access to medical specialties and clinicians other than urologists to perform these procedures. The manipulation of a device in the urethra by clinicians who are not trained or who do not routinely operate on the urinary tract requires specialized training and oversight when manipulating the urinary tract with a urologic device such as the T-SPeC® device. Additionally, patients who have stricture disease (narrowing of the urethra due to formation of scar tissue due to prior urethral trauma or disease) require additional care when passing a sound through the urethra even by highly trained urologic surgeons and, depending upon severity of the stricture disease, may be contraindicated for the T-SPeC® procedure and require a percutaneous procedure such as a trocar punch procedure. It is therefore desirable to provide for improved ease and safety for inside-to-outside and outside-to-inside cystostomy.

### SUMMARY OF THE INVENTION

The present invention is directed to a system and associated functionality for providing optical guidance, with conjunctive illumination, for performing percutaneous (through the skin of abdomen) cystostomy and transurethral cystostomy (T-SPeC®) procedures. In particular, illumination and vision are provided to enable practitioners to monitor instrument progress and operation during such procedures, thereby enhancing patient safety regardless of the procedure and type of practitioner involved. Optically guided percutaneous bladed trocar punch procedures and the optically guided T-SPeC® procedure, as well as associated instruments, are described below.

The invention thus includes a system and associated functionality for providing optical guidance, with conjunctive illumination, for performing transurethral procedures such as the T-SPeC® procedure. In particular, illumination and vision are provided to enable practitioners to monitor instrument progress and operation during such procedures, thereby enhancing patient safety regardless of the type of practitioner involved. As noted above, the T-SPeC® practice is evolving from just urologists towards a broader range of practitioners especially in the hospital environment. Given that these medical professionals may not have prior experience nor training for cystostomy procedures, adding illumination and vision to the T-SPeC® instrument will make the procedure safer in the hands of other clinicians that do not have the specific training of urologists. Thus, turning the “Blind” T-SPeC® procedure, with only anatomical feedback, to one that has illumination and vision, will allow for added safety to the T-SPeC® procedure. In addition, to prevent injury, to increase the ease of passage and reduce the procedure time, Physician Assistants (PA), Nurse Practitioners (NP), Emergency Physicians, Intensivists, or Hospitalists may be required to receive special training for the T-SPeC® procedure, to place suprapubic catheters in patients. The use of vision during suprapubic catheter placement with T-SPeC® will dramatically reduce the training required, help satisfy certification of the cystostomy procedure and increase the safety of the procedure. Moreover, the use of T-SPeC® on patients with urethral stricture can be simplified and safer with the addition of having vision during the placement of the instrument's sound, including passage through the prostate and bladder sphincter. Effectively, the T-SPeC®, with vision, combines both the cystostomy and cystoscopy procedures in one. The combination of the T-SPeC® with vision will allow for a cystoscopy to be performed prior to initiation of the surgical tract, allowing for anatomical examination of the bladder for bladder cancer, a contraindication for a cystostomy with T-SPeC®.

The application of adding optics and lighting illumination to a suprapubic percutaneous bladed trocar punch instrument will provide vison for performing the technique and placement of a suprapubic catheter for treatment of bladder dysfunction due to urinary retention, incontinence or fluid management. This system is referred to herein as the SPC Vision™ system (Suprapubic Percutaneous Cystostomy with Vision)

Prior to SPC Vision, the outside-to-inside trocar punch suprapubic catheter placement procedure has been considered a “blind” procedure, in which a trocar is positioned for placement of a catheter track within two finger breadths cranial to the pubic symphysis allowing for placement of the surgical tract into the bladder from outside-to-inside passing through the abdomen, from the skin to bladder dome, as the optimal location of a catheter for bladder drainage and avoidance of piercing the peritoneal cavity and interposing bowel. This procedure is contraindicated for the pediatric population due to the limited urethra inside diameter of pediatric patients, contraindicating use of the T-SPeC® procedure, a transurethral, inside-to-outside procedure further described herein.

With an impassible urethra due to severe stricture disease or other complications, including the pediatric patient, the outside-to-inside percutaneous cystostomy (surgical creation of an opening into the bladder by incision or puncture from the abdomen into the urinary bladder) is a procedure used for placing a suprapubic catheter, for purposes of draining urine from the bladder. An open cystostomy (surgical procedure with scalpel), is used when percutaneous trocar punch cystostomy is contraindicated, usually due to abdomen size such in the morbidly obese patient and a neurogenic bladder, due to difficulty in locating safe passage.

SPC Vision was designed to replace the existing blind percutaneous trocar “punch” suprapubic catheterization placement techniques existing in the market, which have high mortality and morbidity. The blind percutaneous trocar punch devices have an average annual 4.4% mortality rate in the U.S. and 45% complication rate and the open cystostomy is reported to have 1.8% mortality and 30% complication rate.

The incorporation of vision in the percutaneous bladed trocar punch procedure can prevent severe injury by avoiding the peritoneum and puncture of interposing bowel. Serious injury to the patient and death may be avoided with vision guidance. Additionally, having visual confirmation when accessing the bladder will avoid any “through and through” perforation of other organs by inadvertently passing the trocar needle through the back side of the bladder striking the colon, uterus or other organs, with resulting complications requiring medical intervention and potential death.

SPC Vision is a suprapubic percutaneous cystostomy instrument (trocar (hollow needle) access device) with vision allowing passage through the skin, navigating the subcutaneous tissue, visually avoiding the peritoneum cavity and bowel, passing through the fascia layer and entering the bladder wall. Under vision, the clinician may be able visualize the type of tissue in front of the optics system prior to advancing the trocar, thus enabling the clinician to back up and reposition the trocar to avoid piercing bowel. In the event of inadvertent perforation of bowel, the physician will be in a position to immediately surgically repair a bowel perforation and administer antibiotics to prevent serious sepsis infections and potentially avoiding mortality. With the current percutaneous trocar punch devices/procedures, the physician is not initially aware of bowel puncture and thus the source of serious septic infections goes undetected until too late, with resultant mortality (4.4% U.S. average annual mortality rate for this procedure). The blind percutaneous trocar method requires the bladder to be distended (filled with water) to allow for ease of puncture. Bladder distention is extremely painful to which general anesthesia is required for patient pain management and over distention can result in Acute Kidney Injury (AKI). Once inside of the bladder, the bladed trocar is removed leaving a cannula in-place with the camera in position allowing placement of a guidewire or small diameter catheter in the bladder. Then the cannula is removed, leaving the guide wire or catheter in situ.

SPC Vision, was designed to replace the existing blind trocar “punch” suprapubic placement technique used in urology practices and for hospital critical care patient bladder and fluid management. The addition of vision to the bladed trocar punch device will allow for a safer technique in the hands of trained clinicians performing the procedure when the passage of the urethra is contraindicated for suprapubic placement via an inside-to-outside cystostomy of the Transurethral Suprapubic endo-Cystostomy (T-SPeC®) instrument, the only other safe placement option for suprapubic bladder management. In addition, this new device will also be available for pediatrics, which are contraindicated with the T-SPeC® procedure due urethral size of pediatric patients.

The clinical focus is now for replacement of urethral catheterization with suprapubic catheterization in the hospitalized patient, with a primary focus on the critical care unit (CCU) patient population, which historically receives urethral catheterization at a 70% rate. Over 25% of the general admitted hospitalized inpatient population receive urethral catheters. Urethral catheters are the single largest source of hospital acquired infections world-wide. Because of the proven clinical benefits of suprapubic catheterization, which include elimination of common serious complications due to urethral catheter placement, including urethral perforation, prostate and bladder sphincter injuries, and resultant high infection rates. Suprapubic catheterization is now available to be placed safely, to avoid these serious infections and complications associated with urethral catheterization. Additionally, many blind suprapubic trocar punch catheters are placed in the Emergency Department in patients with severe retention due to an impassable urethra when urgent aspiration is required. Severe, prolonged retention can result in kidney failure, resulting in death.

In accordance with one aspect of the present invention, a medical instrument and associated functionality (“system”) are provided for enabling real-time visualization of a cystostomy involving penetration of a patient's bladder. The medical instrument includes an instrument body, an imaging unit including an optical element, and a light source. The instrument body has a proximal portion and a distal portion and the optical element is directed forwardly from the distal portion of the instrument body for providing image information concerning a volume forward of the distal portion of the instrument body. The light source provides illumination to assist in the imaging of the volume. The medical instrument is connected to a display device for displaying images based on the image information. A practitioner can use the display device to monitor a cystostomy in real time and discern the proper pathway for the surgical tract.

For example, the cystostomy may involve moving the medical instrument on a procedure pathway between a first position, where the distal portion is outside of a bladder (inside tissue when transversing from abdomen to bladder) of a patient and a second position where the distal portion is inside of the bladder. In connection with an outside-to-inside procedure, the medical instrument may include a bladed trocar (hollow needle or sharp-pointed surgical instrument, used with a cannula) that is employed to form a surgical pathway from the abdominal wall of the patient into the patient's bladder. For an inside-to-outside procedure, a medical device including a retractable cutting tool may be introduced into the bladder transurethrally. The cutting tool may then be employed to form a surgical pathway from inside the patient's bladder to the patient's abdominal wall. In either case, advancement of the instrument on the surgical pathway may be monitored in real time, for example, to confirm positioning of the medical instrument and avoid the patient's bowel or other tissue that may pose hazards. As a further alternative, the medical instrument may be employed to monitor a separate device. For example, the medical instrument may be positioned within the patient's bladder (e.g., transurethrally or suprapubically). Then, the medical instrument may be employed to monitor a separate device, such as a percutaneous bladed trocar, dilation tool or cannula, used to form a surgical pathway into the bladder from the abdomen. In this manner, the medical instrument can confirm that the bladed trocar, dilator, catheter, or cannula has been properly positioned within the patient's bladder.

The inventive system may further involve a supply line supported on the instrument body for providing a supply of a substance at said distal portion of said instrument body. For example, the substance may comprise a fluid such as water for flushing a procedure pathway. In this regard, the supply line may include a lumen, extending through the instrument body to the distal portion, for directing a flow of water forwardly of a distal end of the instrument body. The lumen may be connected to a supply of water such as a syringe, water bag, gravity fed from an IV pole, a tank, or water tap via a valve. In this manner, a supply of water can be provided to clear tissue from the optical element and otherwise enhance imaging. Alternatively, the substance may comprise a gel delivered in the vicinity of the optical element to enhance imaging. For example, the gel may have an index of refraction that substantially matches physiological fluids in the surgical pathway.

The medical instrument may be employed in connection with a variety of cystostomy procedures. Such procedures may include imaging procedures, surgical procedures, treatment procedures, and placement of a catheter into the bladder. In the last regard, in connection with an optically guided percutaneous cystostomy, the medical instrument may be inserted from the patient's abdominal wall, through intervening tissue, and into the patient's bladder, all under real-time optical guidance. A bladed trocar may then be withdrawn while a cannula remains in place along the surgical pathway so as to enable insertion of a guidewire through the cannula into the bladder, again with the assistance of real-time imaging. The guide wire can then be used to insert a catheter into the patient's bladder and the guide wire can thereafter be removed. Alternatively, the catheter may be threaded through the cannula prior to withdrawal of the cannula from the patient's bladder. In any case, once the catheter is positioned within the patient's bladder, a balloon can be inflated from the catheter to prevent unintended withdrawal of the catheter from the patient's bladder. This configuration will allow treatment of adult patients with an occluded urethra, as well as pediatric patients where T-SPeC® device is contraindicated.

In accordance with another aspect of the present invention, an apparatus is provided for use in transurethral medical procedures. The apparatus includes a sound and an optical unit, with illumination integrated into the tip of the T-SPeC® sound. Specifically, the sound includes a distal portion, a proximal portion, and at least one handle for gripping by a user to guide the distal portion of the sound through the urethra and into the bladder of a patient. The tip is at the end of the distal portion. The optical unit includes optics, with illumination, directed forwardly from the tip of the sound, for use in providing image information for a volume forward of the sound and one or more light sources directed forwardly from the tip of the sound for use in lighting the volume forward of the sound. The vision apparatus, with integrated illumination, includes and would be connected to a display device, operatively associated with the optical unit, for displaying an image of the volume forward of the sound based on the image information and a communications link, connecting the optical unit to the display, for use in transmitting the image information from the optical unit to the display device. In addition, the images and or video can be retained for further evaluation and or documentation in patients' records. The T-SPeC® instrument with integrated optics/illumination for vision would be connected by wire or wirelessly to a display for continuous viewing during the procedure, either by direct wire or wireless connection so that others outside of the procedure room, could monitor the procedure for training purposes or for further expertise in diagnosis.

The apparatus may further include a cutting end, movably mounted on the sound, and an alignment guide (Alignment Arm with sharps container). The alignment guide is interconnected to the sound so as to be in alignment with the sound and positioned external to the patient when the sound is inserted through the urethra and into the bladder of the patient. The alignment guide facilitates penetration of the cutting end and a tip of the sound through the bladder and an abdominal wall of the patient. In this manner, the distal portion of the sound may extend through the bladder and the abdominal wall such that a urinary catheter can be attached to the tip of the sound and drawn back through the abdominal wall into the bladder.

The optical unit may include an image receiver and a lens for directing light from the volume forward of the sound onto the image receiver. For example, the image receiver may be an end of a light guide such as an optical fiber unit or may include a detector for receiving the light and providing an electrical output signal indicative of the light. The communications link may include a physical connection assembly extending from the image receiver to the display and/or may include a wireless portion.

In accordance with a further aspect of the present invention, a method is provided for use in constructing an apparatus for transurethral medical procedures. The method involves providing a transurethral instrument assembly including a sound having a distal portion and a proximal portion and at least one handle for gripping by user to guide the distal portion of the sound through a urethra and into a bladder of the patient, where the distal portion has a tip at an end thereof. An optical unit is mounted on the tip of the sound. The optical unit includes a lens directed forwardly from the tip of the sound, and a light source for providing illumination of a volume forward of the sound. The method further involves providing a display device and connecting the optical unit to the display device via a communications link, wherein the display device is operative for displaying images of the volume forward of the sound based on imaging information transmitted from the optical unit to the display device via the communications link.

In one implementation, the application of optics and lighting includes the placement of an optics lens and light source in the distal sound tip of the T-SPeC® device with the optics and light cable extending through the instrument sound, body and rear handle exiting at the posterior end of the instrument terminating with a connector plug. The posterior connection interfaces with a detachable power supply to transmit the image to a display panel, monitor or screen with a live image. In this manner, the practitioner can monitor procedures in real-time, including passage of the sound through the urethra and into the bladder of the patient, placement of the sound for initiating cutting, progress of the cutting through the bladder to the abdominal wall and, optionally, drawing of the urinary catheter through the passageway from the abdominal wall into the bladder as well as withdrawal of the device from the patient. Another embodiment could include a battery pack and processor, integrated into the rear handle of the T-SPeC® device to wirelessly transmit image data to a receiver connected to a display for real-time viewing. Captured images and video can be retained for training other clinicians prior to live patient procedures, further enhancing physician and clinician training.

### BRIEF DESCRIPTION OF THE DRAWINGS

For a more complete understanding of the present invention, and further advantages thereof, reference is now made to the following detailed description, taken in conjunction with the drawings, in which:

FIG. 1 is a side cross-sectional view of a T-SPeC® device with an optical unit in accordance with the present invention;

FIG. 2 shows a cable for connecting the optical unit to a display in accordance with the present invention;

FIG. 3 shows a display for displaying real time images for guiding a T-SPeC® procedure in accordance with the present invention;

FIG. 4 shows an optical unit for mounting on the sound of the T-SPeC® device of FIG. 1;

FIG. 5 shows an alternative display device and connection in accordance with the present invention;

FIGS. 6-7 show an optically guided medical instrument including a cannula assembly, dilatator set and a bladed trocar assembly in accordance with the present invention.

FIGS. 8-25 show a series of steps for executing an optically guided cystostomy in accordance with the present invention.

FIGS. 26-28 show a series of steps to widen or dilate the surgical pathway utilizing the optical tube as a guidewire.

### DETAILED DESCRIPTION

The present invention relates to a medical instrument and associated functionality for performing a cystostomy with optical guidance. Such optical guidance may be provided in connection with inside-to-outside or outside-to-inside procedures for penetrating a patient's bladder. A system for executing an inside-to-outside procedure is first described below, followed by a description of an exemplary system for performing an outside-to-inside procedure. While these systems illustrate two important categories of cystostomy, it will be appreciated that other cystostomy-related procedures with optical guidance are possible in accordance with the present invention. Such procedures include procedures for imaging, diagnosis, treatment, training, catheter placement, and stone extraction, among other things. Accordingly, it will be appreciated that the instruments and functionality described herein should be understood as illustrative and not by way of limitation.

Referring to FIG. 1, a T-SPeC® device 10 with an optical unit is shown. The details of the device 10, and the operation thereof, is generally described in U.S. Pat. No. 8,574,256 which is incorporated herein by reference. Generally, the device 10 includes a sound 12 including a distal portion 18 and a proximal portion 20, a front handle 14 and a rear handle 16. A physician or other user can grip the handles 14 and 16 to guide the distal portion 18 of the sound 12 through the urethra and into a bladder of the patient. More specifically, the user guides the tip 22 of the sound 12 to the dome of the bladder, adjacent to the pubic symphysis in preparation for cutting a pathway through the bladder and the abdominal wall of the patient. Conventionally, proper positioning of the tip 22 of the sound 12 has been achieved due to the geometry and features of the device 10 together with the skill of users employing tactile feedback. However, as will be understood from the description below, the illustrated device 10 enables optical guidance to monitor proper positioning visually.

The illustrated device 10 further includes an alignment guide 24. The alignment guide 24 includes an alignment housing 30 mounted on an alignment guide arm 26. The alignment guide arm 26 is slidably mounted on a mast 28 extending from the front handle 14. As described in detail in the above noted U.S. Pat. No. 8,574,256, the device 10 is configured such that the housing 30 is axially aligned with the distal portion 18 of the sound 12, when the distal portion is inserted through the urethra and into the bladder of the patient and is positioned exterior to the patient against the patient's abdominal wall. When the distal portion 18 of the sound 12 is positioned against the dome of the patient's bladder, a cutting end can be deployed from the sound 12 to cut a pathway through the bladder and abdominal wall of the patient. In this regard, the housing 30 provides back pressure against the patient's abdominal wall to facilitate the cutting process.

Once the tip 22 of the sound 12 passes through the patient's abdominal wall, the alignment guide 24 can be removed from the device 10. Then, a urinary catheter can be attached to the tip 22 of the sound 12 and the user can manipulate the sound 12 to draw the tip 22 together with the end of the urinary catheter into the patient's bladder. As discussed above, proper positioning of the sound 12 is required at the various stages of this process. For example, proper positioning is required as the sound 12 passes through the patient's urethra, as the tip on 22 is positioned against the dome of the patient's bladder, as the cutting end is used to penetrate the patient's bladder and abdominal wall, as the urinary catheter is drawn through the abdominal wall and into the bladder, and as the sound 12 is withdrawn from the patient. In accordance with the present invention, proper positioning throughout this process is facilitated by optical guidance.

The illustrated device 10 includes an optical unit 32 mounted at the tip 22 of the sound 12. The optical unit 32 generally includes a distal sound tip cap 34 with a lens opening and an optical lens 36 mounted on the opening. Although not shown in FIG. 1, the optical unit 32 may further include one or more illumination sources, such as LEDs, for illuminating a volume forward of the tip 22 of the sound 12; that is, a volume extending along axis 38 away from the tip 22 towards the housing 30 as illustrated in FIG. 1. The lens 36 is operative to direct light from the illuminated volume onto an end of an optical cable 16. In this regard, the distal end of the cable 16 may be registered in relation to the lens 36 such that light from the lens 36 is received within the acceptance angle of the cable 16. Thus, in the illustrated embodiment, the cable 16 may be a light guide such as a fiber-optic cable. Alternatively, a detector may be disposed between the lens 36 and the cable 16 to detect light and provide an electrical signal representative of the detected light.

An optical connector 40 is provided at the proximal end of the device 10 in the illustrated embodiment. The connector 40 may include an optical detector for detecting light transmitted through the cable 16 and providing an electrical signal representative thereof. In addition, the connector 40 may provide electrical power through the cable 16 to the illumination sources of the optical unit 32. As will be described below, and electrical cable and/or a wireless connection may be used to connect the connector 40 of the device 10 to a display and/or another power source.

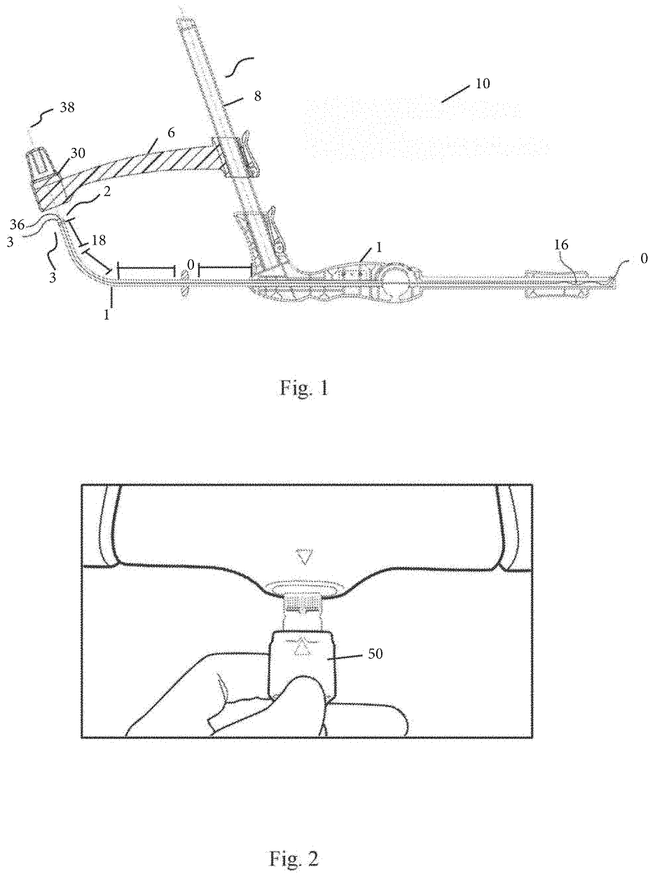

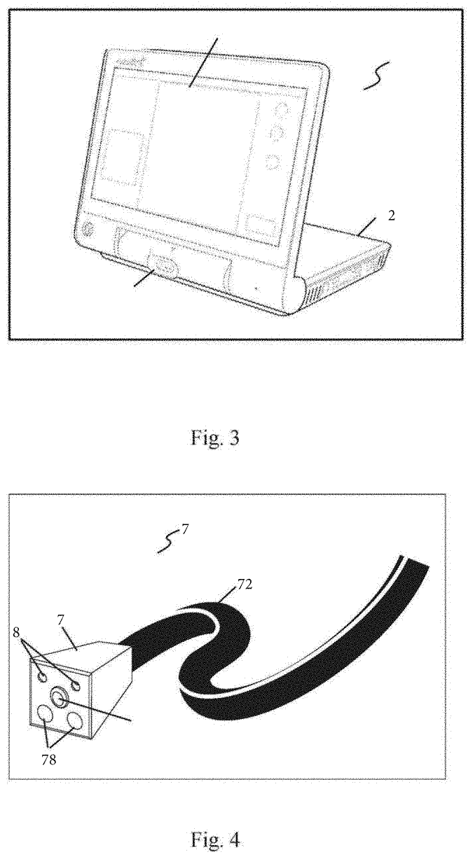

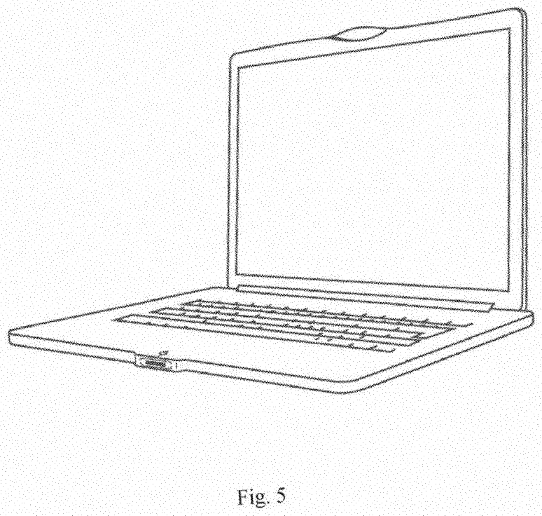

FIG. 3 illustrates a display device 60 that may be used to display images captured by the optical unit for guiding a transurethral medical procedure. The illustrated display device 60 includes a base 62 and a display 64 pivotally mounted on the base 62. The display 64 provides real-time video images of the volume forward of the sound based on information transmitted from the optical unit. The display 64 can be pivoted to an optimal viewing angle for viewing by the user as the user is guiding the sound during the transurethral procedure. The illustrated device 60 further includes a cable port 66 for connecting the display device 60 to the T-SPeC® device. As shown in FIG. 2, a cable 50 can be plugged into the port 66 at the display device and also plugged into a connector of the T-SPeC® device associated with the optical cable. In this regard, the cable 50 can convey imaging information from the optical device to the display device 300 and can also provide electrical power and control signals from the display device 60 to the optical unit, e.g., to turn on and off the optical unit. FIG. 5 shows an alternative display device and connection.

FIG. 4 shows an optical unit 70 that may be mounted on the tip of the sound. The optical unit generally includes a cable 72 having a distal end thereof mounted in a proximal end of housing 74. A lens 76 is mounted on a distal end of the housing 74 in alignment with the distal end of the cable 72 such that light from the lens 76 is received within the cable 72. It will thus be appreciated that the housing 74 maintains the alignment and spacing between the lens 76 and the distal end of the cable 72. Captures imaging information for a volume in front of the lens 76. For example, clear images may be provided on the display of a volume between about 5-100 mm in front of the lens and having a diameter at least equal to that of the sound. The optical unit 70 may further include light sources 78 and 80 disposed about the lens 78 for illuminating the volume to be imaged. For example, the sources 78 and 80 may comprise LEDs and may have the same or different illumination parameters, e.g., power, wavelength and the like. Optionally, the sources 78 and 80 may be individually operable to provide the desired lighting. The optical unit 70 may be mounted on the sound in any appropriate manner such as, for example, an adhesive or sonic welding. In this regard, a complementary mounting structure may be provided on the sound to ensure proper mounting positioning and alignment. It will be appreciated that the dimensions of the optical unit 70 allow for mounting on the sound without interfering with operation of the cutting end and other components of the T-SPeC® device.

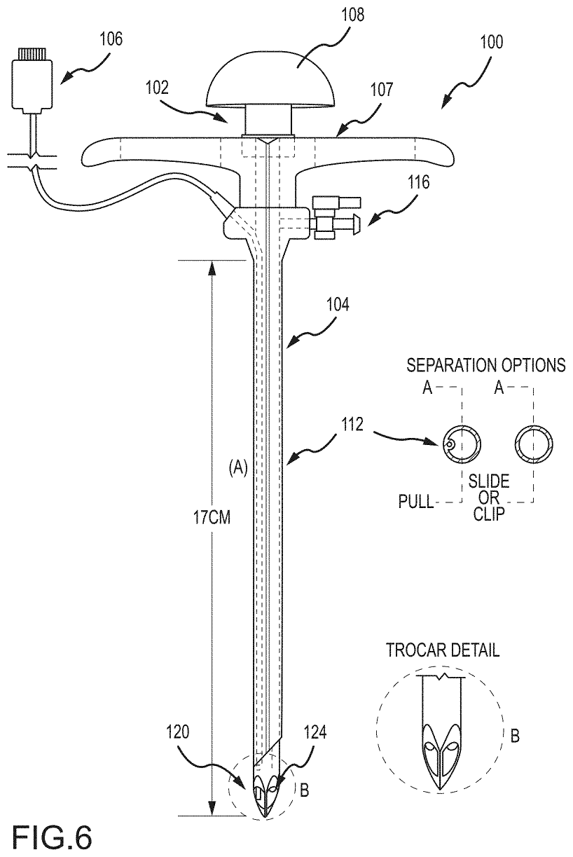

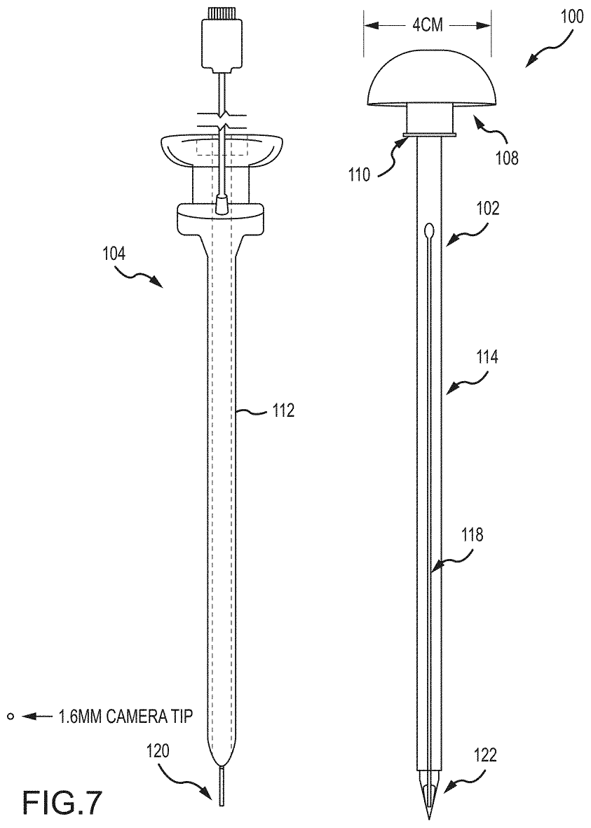

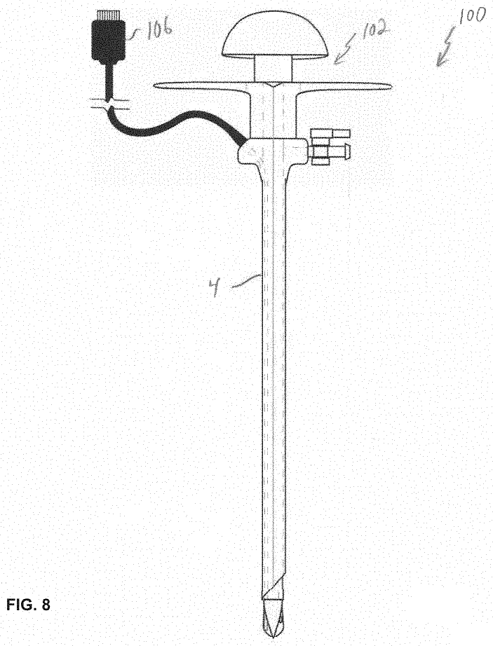

FIGS. 6-7 illustrate an optically guided cystostomy medical instrument 100 in accordance with the present invention. The illustrated instrument includes a bladed trocar assembly 102 and a cannula assembly 104. FIG. 7 shows the assemblies 102 and 104 separated and FIG. 6 shows the assemblies 102 and 104 assembled. The instrument 100 further includes a communications cable 106 for connecting the instrument 102 a display device, as will be described below, and a water valve 116 for connecting the instrument 100 to a supply of water such as a syringe, a tank, water bag, gravity fed from an IV pole, or water tap.

The cannula assembly 104 includes a cannula body 112, an optical unit 120, and the communications cable 106. The cannula housing 112 has a proximal portion adjacent the cable 106 and a distal portion adjacent the optical unit 120. The body 112 defines a hollow internal passageway for receiving the bladed trocar assembly 102. Appropriate connectors extend between the optical unit 120 and the cable 106 for transmitting optical information from the optic unit 120 to the cable 106, e.g., within the hollow center of the cannula body or within the walls of the body 112. The optical unit 120 captures real-time images of a volume forward of the distal end of the instrument 100. These images can be used to monitor the progress of the instrument as the instrument is used to form a surgical pathway from the patient's abdominal wall to the bladder. Simultaneously, video and pictures can be captured and stored for further review, while providing for archival of patient records. In this regard, the optical unit 120 may include one or more LEDs for providing illumination and imaging equipment. For example, the imaging equipment may include a miniature camera unit such as a camera tip or other optical elements, such as one or more lenses and optical fibers for use in transmitting optical information to a remotely located sensor or camera. In the illustrated embodiment, the optical unit 120 includes an LED and a 1.6 mm camera tip. It will be appreciated that other sizes for the camera tip, including smaller dimensions are possible and potentially desirable.

The bladed trocar assembly 102 includes a trocar blade tip 122, a trocar obturator body 114 and an obturator handle 108. The trocar blade tip 122 has a sharp, pointed end for penetrating tissue and is configured to surround the optical unit 120 as well as a water port 124. The trocar obturator body 114 extends between the trocar blade tip 122 and the obturator handle 108 with a groove 118 to accommodate the optical unit 122 housing internal to the cannula body 112. Alternatively, the optical unit 122 (including the fiber optic cable thereof) may be positioned directly into the obturator body/trocar assembly (proximal end) with the distal end in the same position as shown in FIG. 6. In such a case, when the physician removes the obturator/trocar to position a catheter as described below, the operator can then slip the fiber optic cable could be removed from the obturator and slipped down the cleared cannula body. The body 114 has a generally cylindrical configuration and includes an interior water lumen for delivering water from the valve 116 to the water port 124. It will be appreciated that the valve 116 can be opened to allow water to flow from the water port 124 to flush the surgical pathway and improve imaging or closed to terminate water flow. The obturator handle 108 rotates freely on the body 114 and can be gripped by a practitioner to advance or retract the body 114 and trocar blade tip 122 in relation to the cannula assembly 104. The illustrated trocar assembly 102 further includes a fluid seal 110 that forms a seal against the finger pull 107 of the cannula assembly 104 to prevent flow of water or other fluids at the proximal end of the cannula assembly 104 when the handle 108 is fully depressed.

It will be appreciated that the dimensions of the various components of the instrument 100 may vary in accordance with the present invention. In the illustrated embodiment, the handle 108 has a width or diameter of about 4 cm. The instrument 100 may have a length of about 17 cm from the distal end of the trocar assembly 102 to a collar area of the cannula assembly 104 when the trocar assembly 102 is fully inserted. The cannula may have a range of sizes with an outside diameter ranging from of 14 to 28 French, dependent on patient size and need. Moreover, as shown in FIG. 1 and described in more detail below, the cannula may be formed in two pieces to separate along a longitudinal axis of the cannula. This may be desired, for example, to enable removal of the cannula after a catheter has been installed via the cannula. In this regard, the cannula assembly 104 may be configured to separate by pulling on opposite halves of the cannula assembly or by operating a slider or clip to release the two halves.

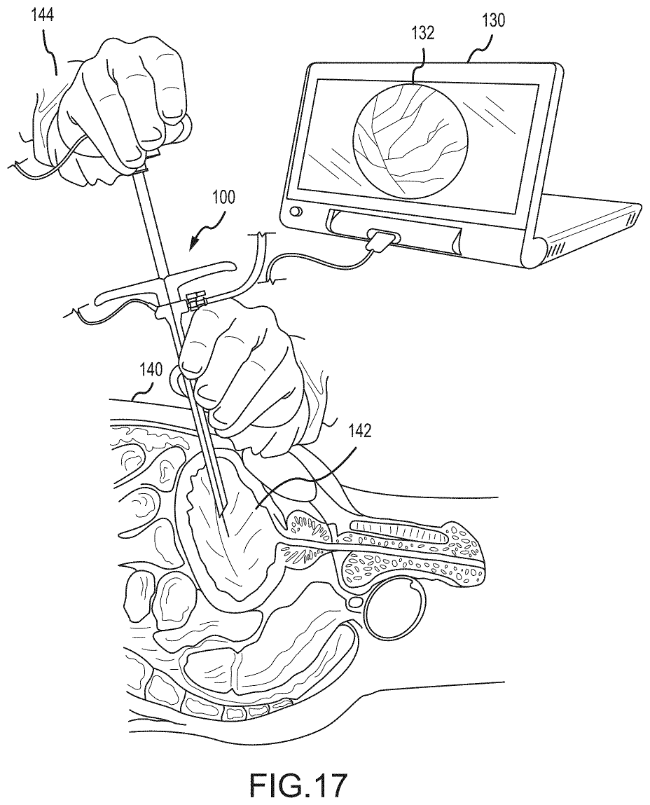

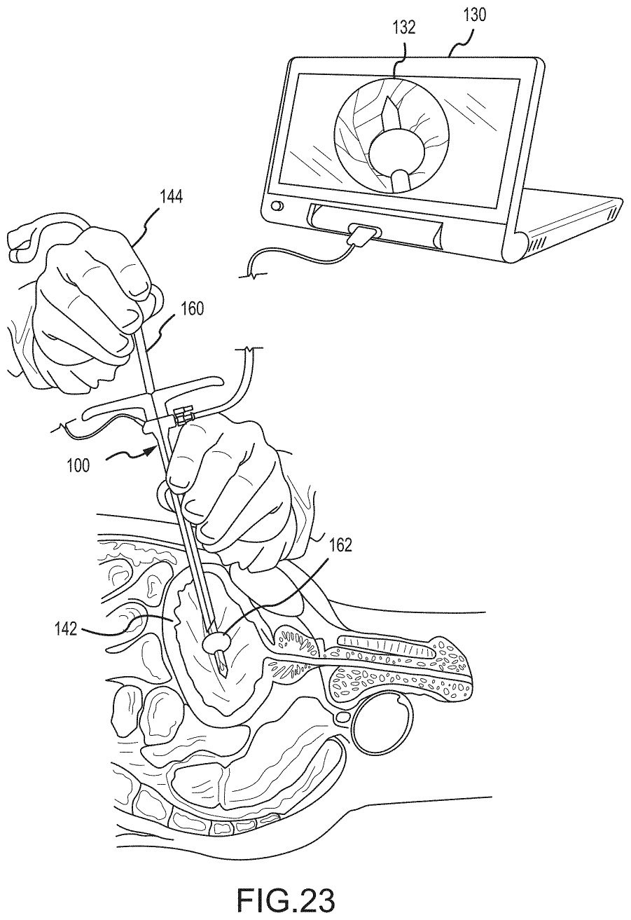

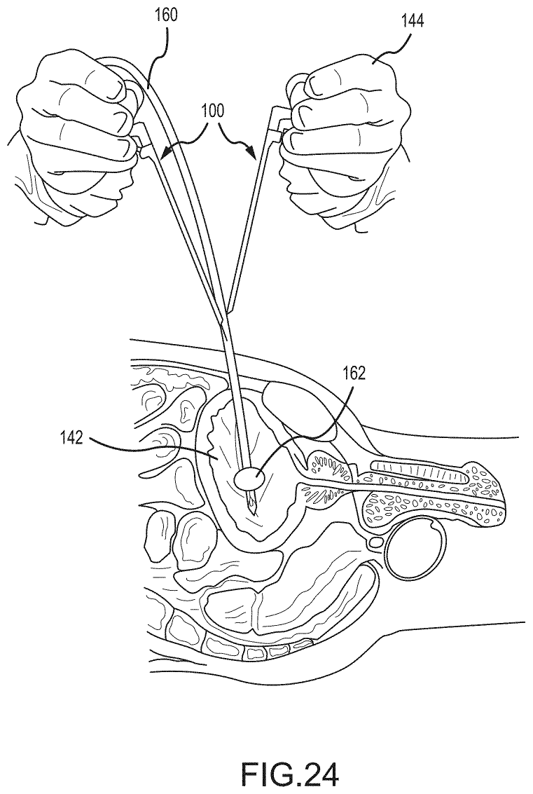

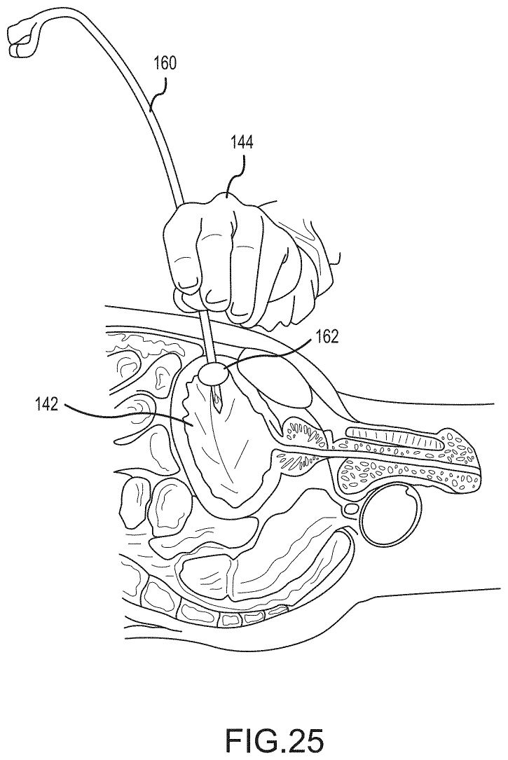

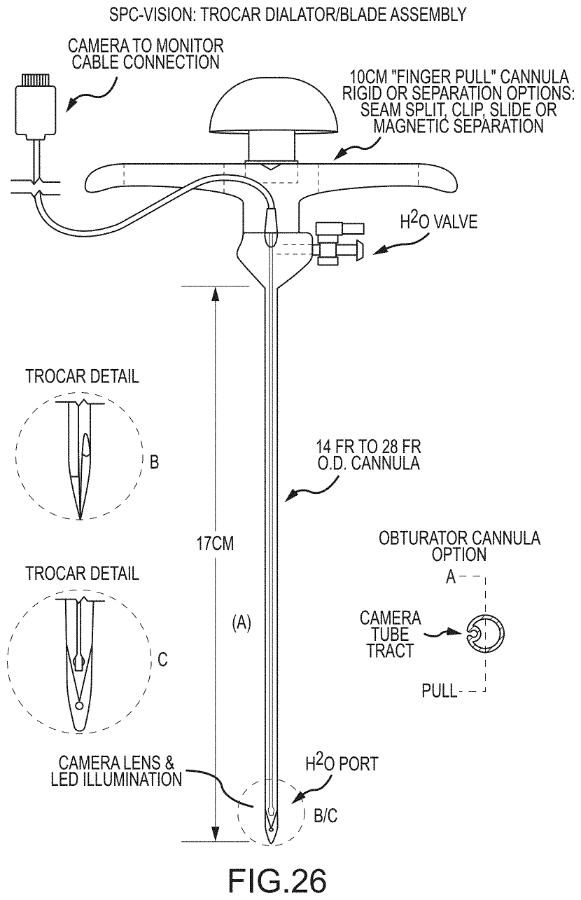

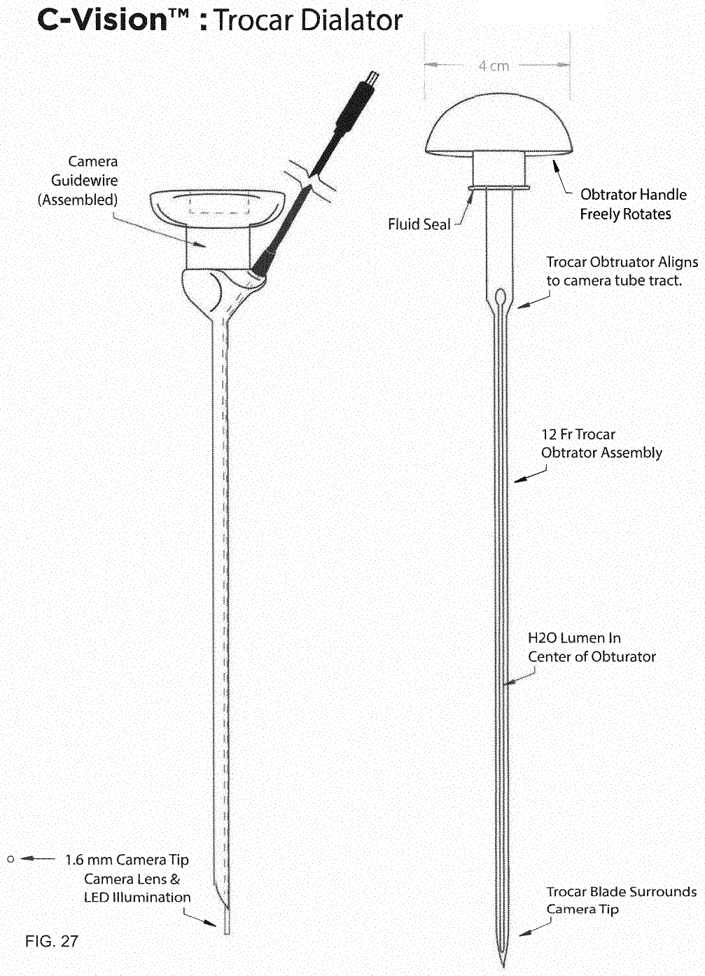

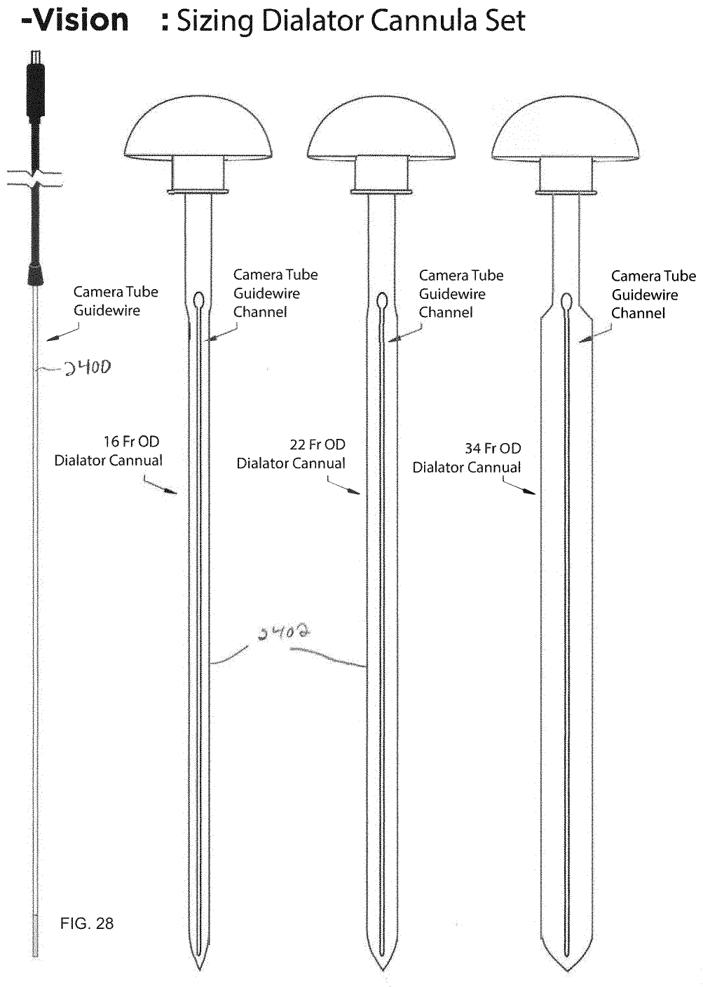

FIGS. 8-25 show an illustrative process, in accordance with the present invention, for forming a surgical pathway to a patient's bladder and inserting a catheter or Dilation Cannula all under optical guidance. By way of overview, the percutaneous suprapubic cystostomy with SPC Vision™ procedure steps include:
1) Connect SPC Vision, device to vision display monitor
2) Connect water supply to SPC Vision, device for improved visualization
3) Prep skin surface above pubic symphysis with localized antiseptic
4) Palpate patient's abdomen to locate the pubic symphysis
5) Advance cannula with trocar at two finger breadths above pubic symphysis through skin into the bladder, visualizing passageway
6) After locating the cannula and trocar within the bladder, remove the trocar from cannula, leaving cannula in situ
7) Option 1. A Guidewire is placed through Cannula. Then remove Cannula and place catheter over Guidewire or “size-up” the surgical tract for a larger catheter, requiring further tract dilation, utilizing balloon dilation. Then place Catheter over Guidewire and complete procedure.
   a. Aspirate urine through Foley catheter to confirm placement
   b. Set Catheter at dome of bladder
   c. Dress the site
8) Option 2. Advance a small-bore catheter through the cannula and into the bladder.
   a. Aspirate urine through Foley catheter to confirm placement
   b. Inflate Foley catheter balloon
   c. Remove Cannula and withdraw from patient
   d. Dress the site
9) Option 3 (FIGS. 26-28). Advance dilation cannula over the optics tube as a “guide wire” to size-up the surgical pathway for desired use
   a. Repeat with increased diameter of dilation cannulas 2402 (16, 22, and 34 French shown in FIG. 28) as needed
   b. Perform surgical procedure or placement of catheter through dilation cannula
   c. Inflate Foley catheter balloon
   d. Remove dilation cannula and withdraw from patient
   e. Dress the site

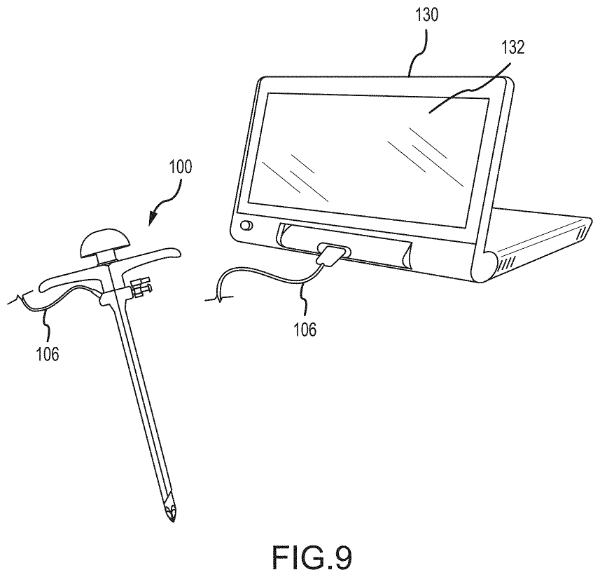

FIG. 8 shows a medical instrument for use in the procedure which may be substantially as described in connection with FIGS. 6-7. As shown in FIG. 9, the process may be initiated by connecting the communications cord 106 to a display device 130 including a monitor 132. The display device 130 may include, for example, a laptop computer, a dedicated display device for the system, or another display device available in the procedure environment.

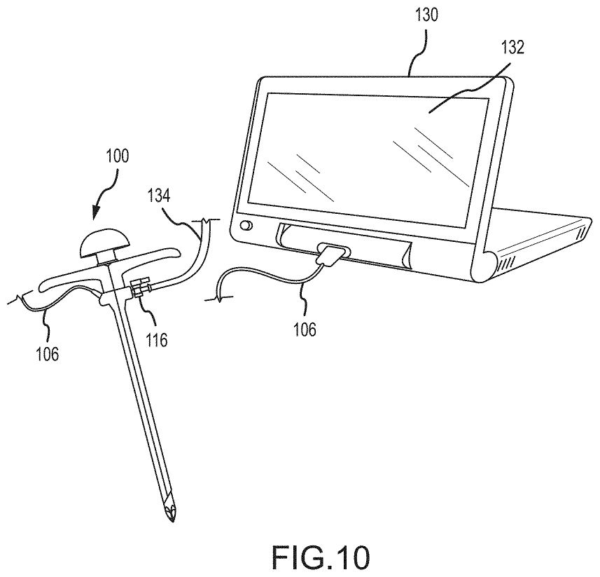

Next, a water supply 134 may be connected to the valve 116 (FIG. 10). Although not shown, it will be appreciated that the water supply 134 may be connected to a water source such as a water tank or water tap. It will be appreciated that the valve 116 may be operated to provide water at the distal end of the instrument 100 when needed to flush the surgical pathway or otherwise improve imaging.

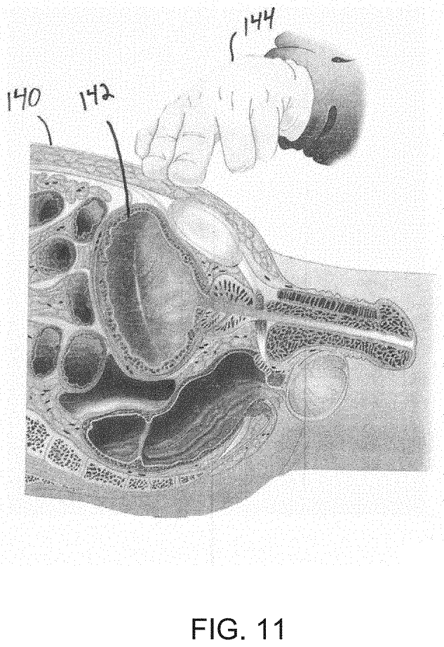

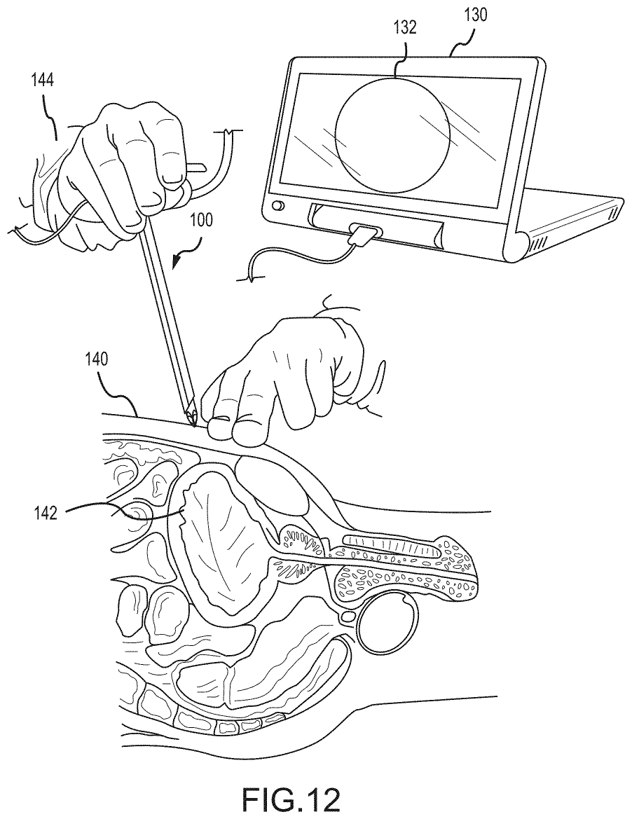

As noted above, it is important to initiate an incision to access the patient's bladder at the proper location on the patient's abdominal wall. In this regard, it is generally desired to form the surgical pathway above the pubic symphysis while avoiding the peritoneum cavity and bowel so as to access the dome of the bladder. As shown in FIGS. 11-12, a practitioner 144 may locate the pubic symphysis via palpation and then initiate incision of the skin/abdominal wall, using the instrument 100, about two finger breadths cranial to the pubic symphysis.

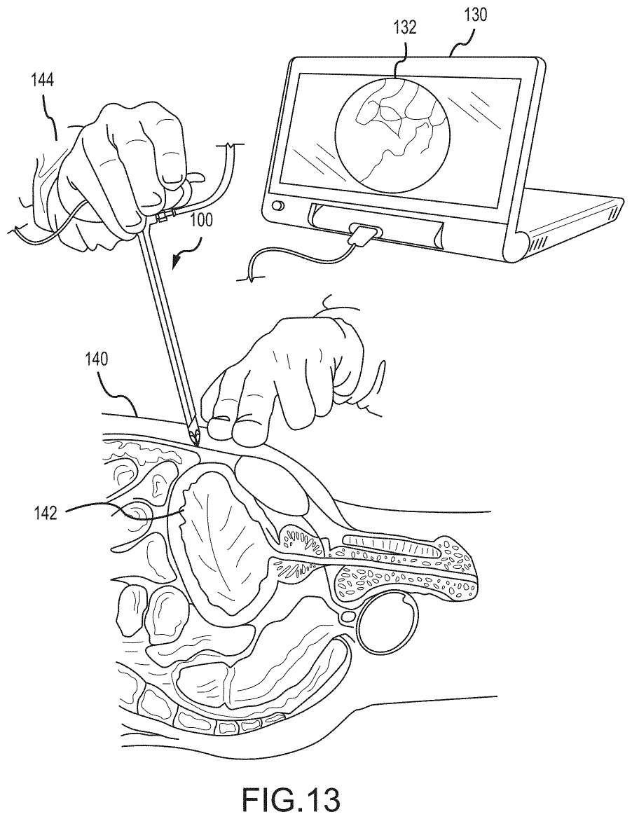

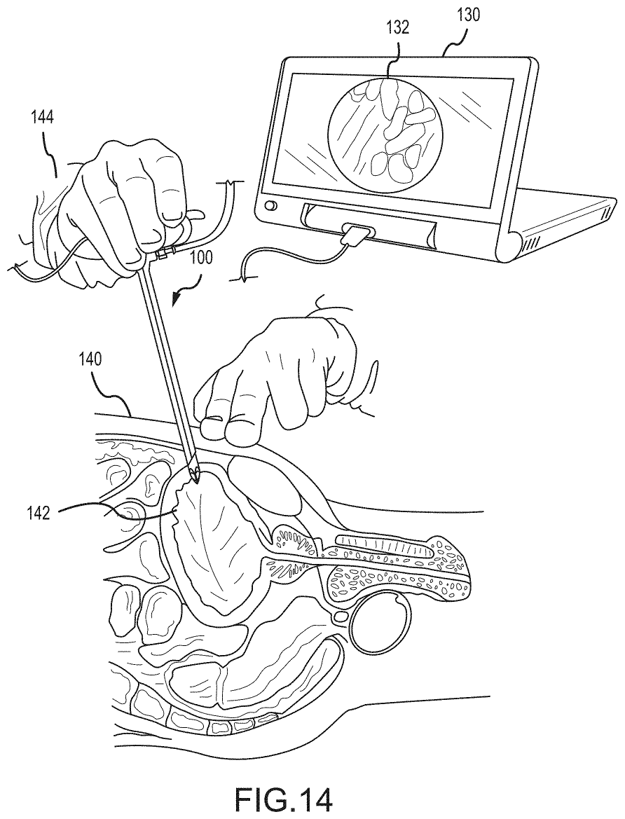

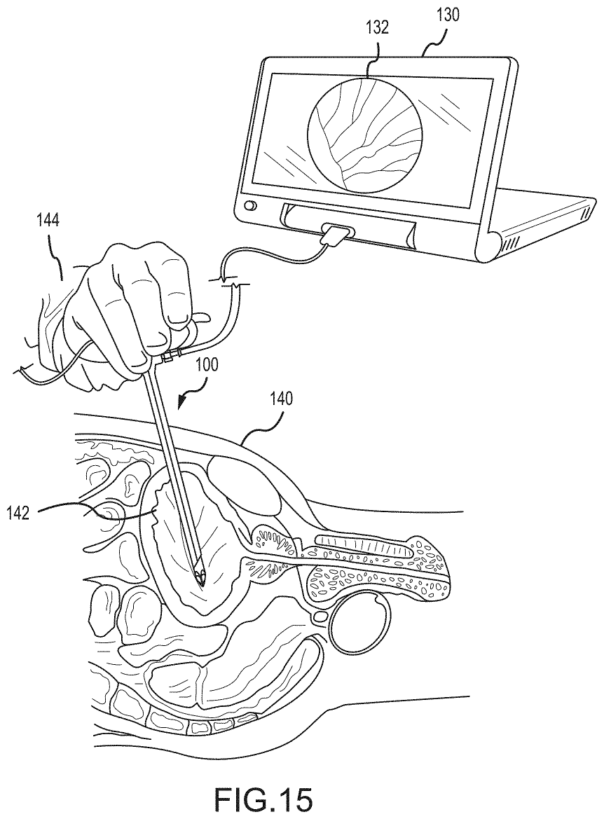

The practitioner 144 can then view the imaging device 130 to monitor advancement of the distal end of the instrument 100 through the skin and fatty tissue (FIG. 13), through the subcutaneous tissue and fascia layer, and through the bladder wall into the interior of the bladder (FIGS. 14-15).

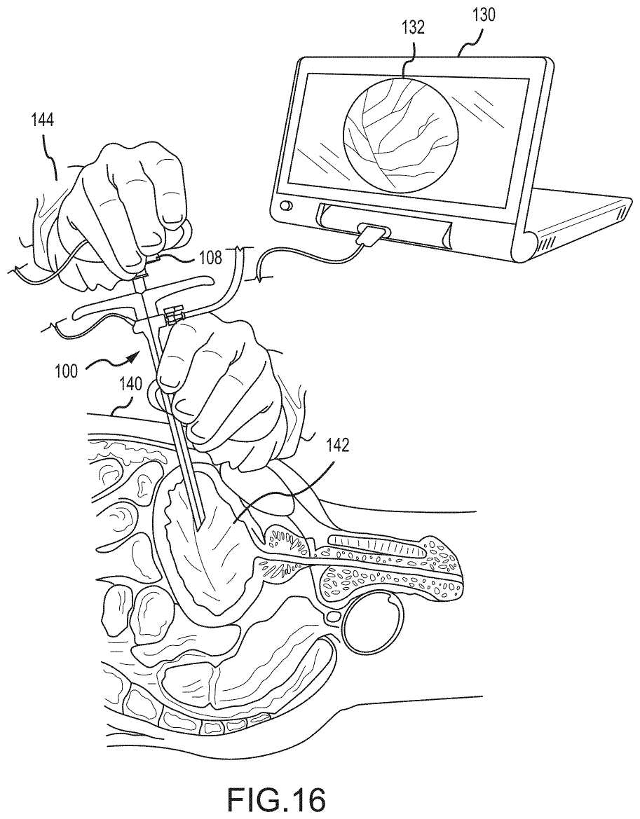

Once the distal end of the instrument 100 is properly positioned within the bladder and such positioning is confirmed on the monitor 132, the practitioner 144 may lift the trocar handle 108 while holding the cannula of the instrument 100 in place to remove the trocar assembly 102 as shown in FIGS. 16-18.

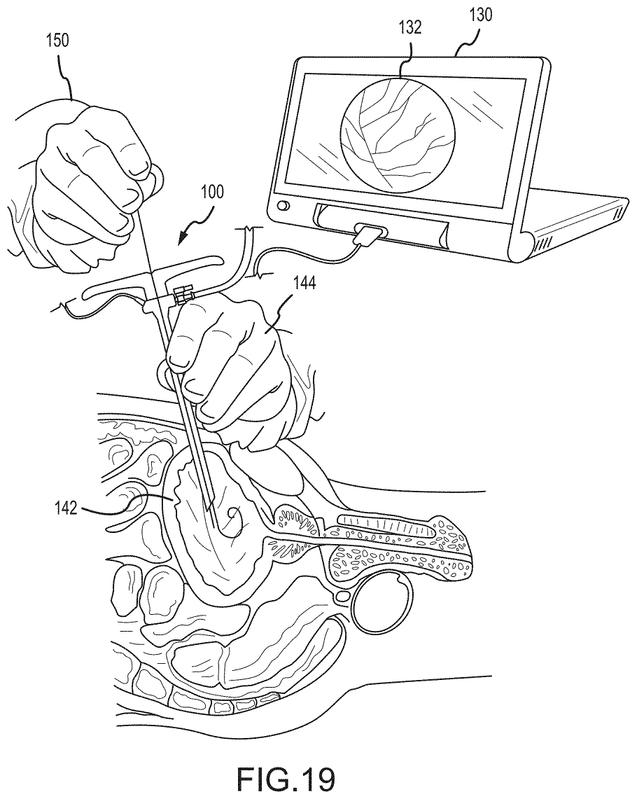

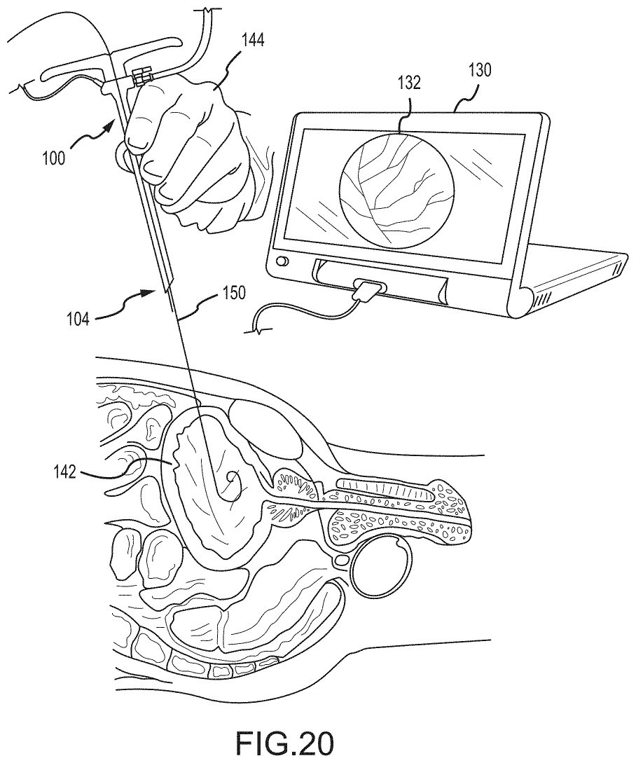

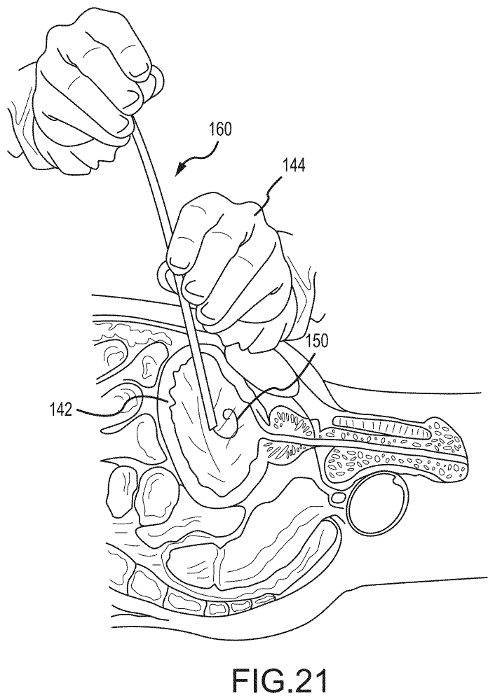

Once the trocar assembly 102 has been removed from the instrument 100, there are a couple of options for proceeding with insertion of a catheter. In a first option, as illustrated in FIGS. 19-21, a guide wire 150 is first inserted through the cannula assembly of the instrument 100 into the bladder 142. As shown in FIG. 19, proper positioning of the guide wire 150 within the bladder 142 can be confirmed on the monitor 132. The instrument 100 can then be withdrawn leaving the guide wire 150 in place (FIG. 20). As shown in FIG. 21, a catheter 160 can then be threaded over the guide wire 150 and through the surgical pathway formed by the instrument 100 so that the distal end of the catheter 160 is positioned within the bladder 142. The guide wire 150 can then be withdrawn through the catheter 160.

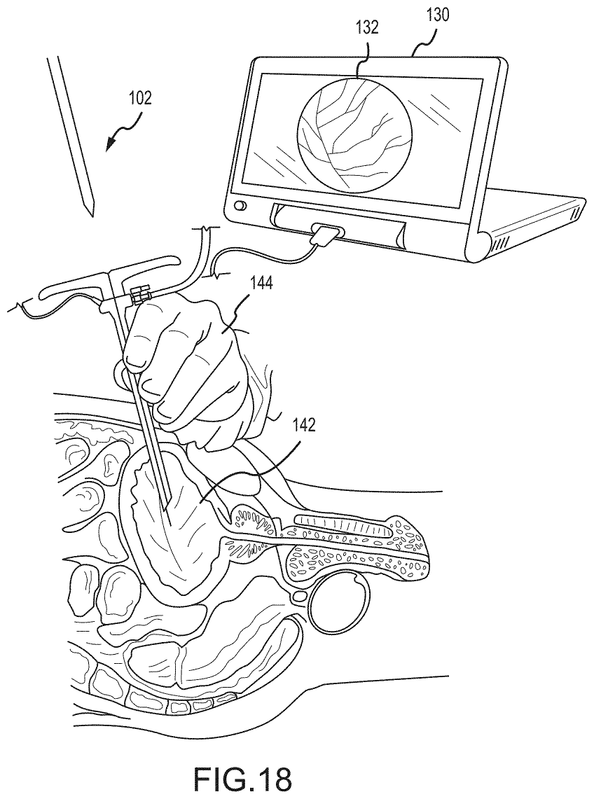

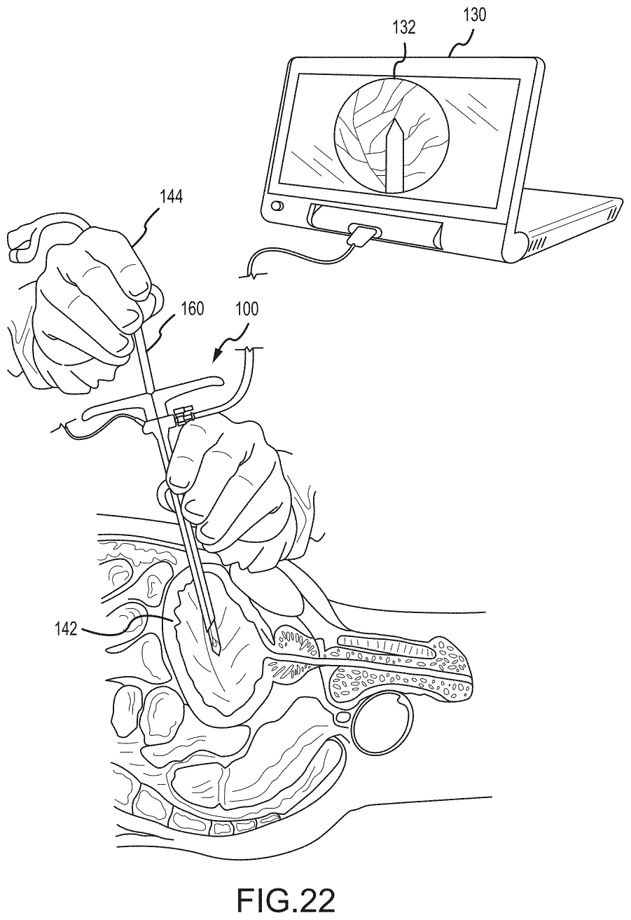

Alternatively, as shown in FIG. 22, the catheter 160 may be threaded through the instrument 100 so that the distal end of the catheter 160 is disposed within the bladder 142 as confirmed via the display device 130. A balloon 162 near the distal end of the catheter 160 may then be inflated to prevent unintended withdrawal of the catheter 160 from the bladder 142 (FIG. 18). As shown in FIG. 19, the instrument 100 may then be withdrawn from the patient and longitudinally separated into halves to remove the instrument 100 from the catheter 160. Finally, the practitioner 144 may withdraw the catheter 160 until the balloon 162 is secured against the wall of the bladder 142. The practitioner 144 can then dress the incision to complete the procedure.

The SPC Vision device and technique is an enabling technology for replacing urethral catheterization with suprapubic catheterization in hospitalized patients with occluded urethras, when inside-to outside suprapubic catheterization (SPC) is contraindicated with the T-SPeC® instrument, allowing these patients access to SPC, further improving clinical outcomes, while simultaneously saving hospitals money due to reduced length of stay, all realized with this change in clinical practice. The utilization of SPC, replacing usage of urethral catheterization in select patients will reduce extended hospital stays, readmissions and complications associated with indwelling urethral catheters. The rationale for integrating illumination and visual guidance with the SPC Vision device is to replace the “blind” entry to the bladder when an inside-to-outside transurethral suprapubic cystostomy with the T-SPeC® device is contraindicated due to an occluded urethra. Inside-to-outside transurethral suprapubic cystostomy with the T-SPeC® device is contraindicated when there is severe stricture disease (urethral scar tissue), obstructing the urethra. Prior to initiating a cystostomy, utilizing an endoscope/cystoscope, may be used to inspect the health of the urethra passage and bladder, for detecting possible bladder cancer, a procedure called a Cystoscopy. Urologists are very familiar with these procedures. If passage of urethra is not available, the alternative outside-to-inside trocar punch technique or highly invasive open cystostomy is required. Failure to void the bladder of a patient in severe retention will lead to acute kidney injury, kidney failure, and mortality.

Due to the acute shortage of urologists, and limited availability to support hospital-based patient catheterization with SPC, mid-level clinicians such as PAs, RNPs and emergency physicians will be utilized to place suprapubic catheters. These clinicians are less experienced with SPC placement, especially with “blind SPC placement techniques where patient safety can be of concern. Thus, adding vision to SPC placement techniques can improve procedure safety. Over 70% of all critical care patients are transferred from the emergency department (E.D.), with the remainder coming from the Med/Surg area. In order to place suprapubic catheters in patients being transferred to the CCU, it is necessary to provide T-SPeC® and SPC Vision procedure training to mid-level clinicians, such as PAs and nurse practitioners and emergency physicians. Furthermore, U.S. men 40 to 83 years of age have an overall incidence of nearly 7 per 1,000 per year present to the E.D. with acute or severe urinary retention and an impassible urethra due to Benign Prostatic Hyperplasia (BPH), Infection and inflammatory issues or other penile trauma, fracture or laceration. To prevent injury, to increase the ease of passage and reduce the procedure time Physician Assistants (PAs), Registered Nurse Practitioners (RNPs), Emergency Physicians, Intensivists, or Hospitalists will require special training in the use of the SPC Vision procedure and device, to place suprapubic catheters in patients with compromised, occluded urethral passage. The use of vision during suprapubic catheter placement with SPC Vision will dramatically reduce the training required and improve the overall safety of cystostomy in patients contraindicated for urethral SPC placement with the T-SPeC® instrument.

The application of optics and lighting would include the placement of the optics camera lens and light source in the distal sound tip of the SPC Vision, cannula with the camera and light cable extending through the instrument sound, body and handle exiting at the posterior end of the instrument terminating with a connector plug. The posterior connection would interface with a detachable power supply to transmit the image to a display panel, monitor or screen with a live image, with additional capability of storing video and still picture images for retrieval and to be included in the medical records for patient.

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain best modes known of practicing the invention and to enable others skilled in the art to utilize the invention in such, or other embodiments and with various modifications required by the particular application(s) or use(s) of the present invention. It is intended that the appended claims be construed to include alternative embodiments to the extent permitted by the prior art.

1. A method for use in cystostomy, comprising:
providing a medical instrument including an instrument body having a proximate portion and a distal portion, and an optical assembly supported on said instrument body, said optical assembly including an optical element, directed forwardly in relation to said distal portion of said instrument body, for use in providing image information for a volume forward of said distal portion of said instrument body, and a light source, directed forwardly from said distal portion of said instrument body, for use in providing illumination of said volume forward of said distal portion of said instrument body; connecting said medical instrument to a display device for displaying images based on said image information; and
using said display device to monitor a cystostomy.

2. The method of claim 1, wherein said medical procedure involves moving said medical instrument on a procedure pathway between a first position, where said distal portion is outside of a bladder of a patient, and a second position, wherein said distal portion is inside of said bladder.

3. The method of claim 1, wherein said using involves monitoring movement of a device separate from said medical instrument relative to a bladder of a patient.

4. The method of claim 2, wherein said medical instrument comprises a trocar having a cutting end and said medical procedure involves advancing said cutting end from said first position to said second position.

5. The method of claim 2, wherein said medical instrument comprises a boom supporting a retractable cutting blade, and said medical procedure involves employing said cutting blade to advance said boom from said second position to said first position.

6. The method of claim 1, wherein said medical instrument further comprises a supply line supported on said instrument body for providing a supply of a substance at said volume forward of said distal portion of said instrument body. The supply line of water used to cleanse surgical pathway from blood and/or tissue to provide visual acuity, such that tissue identification is not impeded, during advancement of trocar through abdomen into bladder.

7. The method of claim 6, wherein said substance comprises water for flushing a procedure pathway.

8. The method of claim 6, wherein said substance comprises a gel for improving imaging by said optical element.

9. The method of claim 1, wherein said cystostomy involves inserting a guide wire through said medical instrument into said patient's bladder.

10. The method of claim 9, wherein said cystostomy further involves using said (Original) guide wire to guide a further medical instrument to said patient's bladder.

11. The method of claim 10, wherein said further medical instrument comprises a catheter.

12. The method of claim 1, wherein said optical assembly functions as a guide wire for guiding insertion of a further device along a surgical path defined by said medical instrument.

13. The method of claim 12, wherein said further device comprises an obturator for widening said surgical path.

14. An apparatus for use in cystostomy, comprising:
a medical instrument including an instrument body having a proximate portion and a distal portion, and an optical assembly supported on said instrument body, said optical assembly including an optical element, directed forwardly in relation to said distal portion of said instrument body, for use in providing image information for a volume forward of said distal portion of said instrument body, and a light source, directed forwardly from said distal portion of said instrument body, for use in providing illumination of said volume forward of said distal portion of said instrument body, said medical instrument being adapted for performing a medical procedure involving movement of said medical instrument on a procedure pathway between a first position, where said distal portion is outside of a bladder of a patient, and a second position, wherein said distal portion is inside of said bladder;
a display device for displaying images based on said image information.

15. The apparatus of claim 14, wherein said display device is operative for monitoring movement of a device separate from said medical instrument relative to a bladder of a patient.

16. The apparatus of claim 15, wherein said medical instrument comprises a trocar having a cutting end for cutting a pathway from said first position to said second position.

17. The apparatus of claim 15, wherein said medical instrument comprises a boom supporting a retractable cutting blade operative to advance said boom from said second position to said first position.

18. The apparatus of claim 14, wherein said medical instrument further comprises a supply line supported on said instrument body for providing a supply of a substance at said volume forward of said distal portion of said instrument body.

19. The apparatus of claim 18, wherein said substance comprises water for flushing a procedure pathway.

20. The apparatus of claim 18, wherein said substance comprises a gel for improving imaging by said optical element.

21. The apparatus of claim 14, wherein said instrument body is configured for inserting a guide wire through said medical instrument into said patient's bladder.

22. The apparatus of claim 14, wherein said optical assembly functions as a guide wire for guiding insertion of a further device along a surgical path defined by said medical instrument.

23. The apparatus of claim 22, wherein said further device comprises an obturator for widening said surgical path.

24-33. (canceled)
